(19) United States
(12) Patent Application Publication
(10) Pub. No.: US 2024/0211658 A1
(43) Pub. Date: Jun. 27, 2024

(54) SYSTEM AND METHOD FOR SIMULATING A LOCALIZED SURFACE PLASMON RESONANCE(LSPR) SPECTROMETER

(71) Applicant: NICOYA LIFESCIENCES INC., Kitchener (CA)

(72) Inventors: Udayan KANADE, Pune (IN); Sanat GANU, Pune (IN)

(21) Appl. No.: 18/554,670

(22) PCT Filed: Apr. 20, 2022
(86) PCT No.: PCT/CA2022/050602

§ 371 Date: Oct. 10, 2023

Related U.S. Application Data
(60) Provisional application No. 63/177,484, filed on Apr. 21, 2021.

(51) Int. Cl. G06F 30/25; G01N 21/552; G06F 111/10; G16B 5/00

(52) U.S. Cl. CPC G06F 30/25; G01N 21/554; G16B 5/00; G06F 2111/10

(57) ABSTRACT

A system and method for computationally simulating an LSPR spectrometer is described herein. The method includes reading a target peak wavelength, using a mathematical model of an LSPR spectrometer system to compute an absorbance/reflectance spectrum, using a mathematical model of an LSPR spectrometer system and an illumination source spectrum to compute an absorbed/reflected spectrum of optical dispersion, and perturbing the absorbed/reflected spectrum with imaging noise.

### CROSS-REFERENCE TO RELATED APPLICATIONS

This application claims priority to U.S. Patent App. No. 63/177,484, entitled “System and Method for Simulating a Localized Surface Plasmon Resonance (LSPR) Spectrometer,” filed on Apr. 21, 2021, which is incorporated herein by reference.

### TECHNICAL FIELD

The presently disclosed subject matter relates generally to localized surface plasmon resonance (LSPR) spectrometry. More specifically, the invention relates to a system and method for computationally simulating an LSPR spectrometer.

### BACKGROUND

An LSPR spectrometer is a chemical analysis spectrometer in which ligand protein molecules are immobilized onto nanoparticles, such as gold nanoparticles. The molecule to be analyzed, known as the analyte, binds to the ligand and causes a shift in LSPR resonant frequency of the nanoparticle. This resonant frequency is probed using absorbance/reflectance spectrometry, and is seen as a peak in the frequency/wavelength of the absorbance/reflectance. However, currently the information contained in LSPR hardware datasets for LSPR data analysis is limited. For example, LSPR hardware datasets may contain a certain amount of systemic noise (physico-chemical noise).

### SUMMARY

A system and method for computationally simulating an LSPR spectrometer is described herein. The method includes reading a target peak wavelength, using a mathematical model of an LSPR spectrometer system to compute an absorbance/reflectance spectrum, using a mathematical model of an LSPR spectrometer system and an illumination source spectrum to compute an absorbed/reflected spectrum of optical dispersion, and perturbing the absorbed/reflected spectrum with imaging noise.

In one aspect, the present invention is directed to a method for computationally simulating an LSPR spectrometer system, the method comprising: (a) reading a target peak wavelength; (b) using a mathematical model of the LSPR spectrometer system to compute an absorbance/reflectance spectrum; (c) using the mathematical model of the LSPR spectrometer system and an illumination source spectrum to compute an absorbance/reflectance spectrum of optical dispersion; and (d) perturbing the absorbed/reflected spectrum with optical dispersion imaging noise to create a noise perturbed spectrum.

In one embodiment, the noise perturbed spectrum is stored as a 2D image.

In one embodiment, the absorbance/reflectance spectrum is computed using Mie theory. In another embodiment, the absorbance/reflectance spectrum is computed using a log-normal function.

In one embodiment, the optical dispersion imaging noise is modeled using a 2D convolution. In some embodiments, the imaging noise is photon noise.

In one embodiment, the target peak wavelength is computed using a binding kinetics reaction simulator.

In another aspect, the present invention is directed to a method for computationally simulating a binding kinetics reaction, the method comprising: (a) choosing a binding kinetics model and parameters for the binding kinetics model; (b) using the binding kinetics model to compute a binding response as a function of time; (c) discretizing the binding response into a plurality of discrete time instances; and (d) finding the peak wavelength corresponding to each discrete time instant.

In one embodiment, the binding kinetics model is Langmuir 1:1. In another embodiment, the binding kinetics model is Langmuir 1:1 with mass transport limitations. In still another embodiment, the binding kinetics model is Langmuir 1:1 with drift.

In some embodiments, the binding kinetics model is a two-state conformation model. In other embodiments, the binding kinetics model is a bivalent analyte model.

In still other embodiments, the binding kinetics model is a heterogeneous analyte model. In one embodiment, the binding kinetics model is a heterogeneous ligand model. In some embodiments, the binding response is computed using numerical integration of the binding kinetics model.

### 1. Definitions

### 1.1. Acronyms

“AuNPs” refers to “gold nanoparticles.”

“LSPR” means “localized surface plasmon resonance.”

### 1.2. General Definitions

“Absorbance spectrum” means the spectrum of light absorbed by the LSPR gold nanoparticles when a uniform input spectrum is incident on them.

“Core-shell spheres” means a model of coated nanoparticles consisting of a core particle encapsulated by a shell.

“Localized surface plasmon resonance” means the collective oscillation of electrons at the interface of metallic structures.

“Nanoparticles” means particles with one or more dimensions less than 100 nm.

“Particles” means particles with one or more dimensions greater than 100 nm.

“Reflectance spectrum” means the spectrum of light reflected from the LSPR gold nanoparticles, when a uniform input spectrum is incident on them.

### BRIEF DESCRIPTION OF THE DRAWINGS

The features and advantages of the present invention will be more clearly understood from the following description taken in conjunction with the accompanying drawings, which are not necessarily drawn to scale, and wherein:

FIG. 1 illustrates a block diagram of an example of an LSPR spectrometer system for simulating an LSPR spectrometer according to the invention;

FIG. 2 illustrates a block diagram of an example of a mathematical model of an LSPR spectrometer system according to the invention;

FIG. 3 illustrates a flow diagram of an example of a method of computationally simulating a single frame of an LSPR spectrometer system according to the invention;

FIG. 4 illustrates a flow diagram of an example of a method of computationally simulating a frame sequence of an LSPR spectrometer system corresponding to temporal binding of a ligand and an analyte; and

FIG. 5 illustrates a block diagram of an example of an image processor for executing computational simulations in accordance with the invention.

### DETAILED DESCRIPTION OF THE INVENTION

The invention provides a system and method for computationally simulating an LSPR spectrometer. In one aspect, the method includes:
1) reading a target peak wavelength,
2) using a mathematical model of the LSPR spectrometer system to compute the absorbance/reflectance spectrum,
3) creating an absorbed/reflected spectrum of optical dispersion,
4) perturbing the spectrum with imaging noise, and
5) optionally, storing the noise perturbed spectral data as a 2D image.

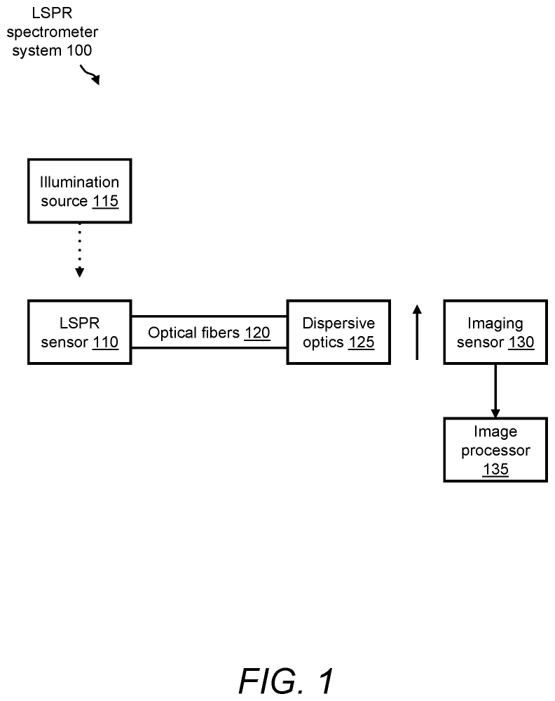

FIG. 1 is a block diagram of an example of an LSPR spectrometer system 100 for simulating an LSPR spectrometer. An LSPR spectrometer is used in the art to determine the chemical affinity between a pair of molecules or bodies, such as proteins, antigens, antibodies, drugs, and the like. The LSPR spectrometer system 100 may include an LSPR sensor 110 having gold nanoparticles (AuNPs) deposited on its surface. In some embodiments, nanoparticles other than gold nanoparticles may be provided on the LSPR sensor 110. One of the bodies to be analyzed, the ligand, is immobilized on the AuNPs while the other body, the analyte, is introduced in the form of a fluid. The binding between the two bodies changes the optical properties of the AuNPs, causing a shift in the peak absorbance and reflectance spectra of the AuNPs. An illumination source 115 may be used to shine light of a known spectrum onto the LSPR sensor 110. The reflected/transmitted light from the LSPR sensor 110 is coupled to optical fibers 120 and channeled to a dispersive optics 125. The dispersive optics 125 includes elements such as a diffraction grating which separates the light from the optical fibers 120 into its constituent wavelengths. This dispersed light then falls on an imaging sensor 130. In one example, the imaging sensor 130 may be a camera. The imaging sensor 130 maps the dispersed light into a 2D image which is analyzed using an image processor 135. The image processor 135 estimates the peak reflectance/absorbance wavelength of the AuNPs in the LSPR sensor 110. In one example, the image processor 135 may be dedicated hardware designed to perform the image processing task. In another example, the image processor 135 may be a computer running a program that performs the computations for estimating the peak reflectance/absorbance wavelength of the LSPR sensor 110.

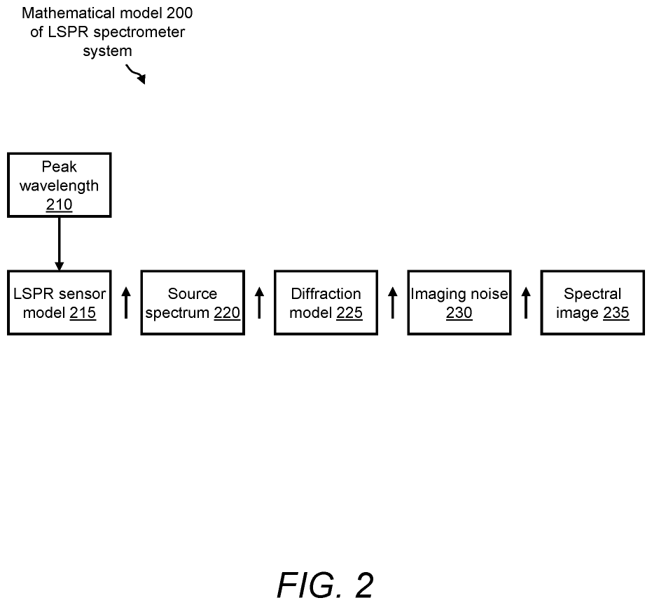

FIG. 2 is a block diagram of an example of a mathematical model 200 of an LSPR spectrometer system, such as the LSPR spectrometer system 100 shown in FIG. 1. A peak wavelength 210 represents the peak wavelength of the LSPR sensor 110 and is an input to an LSPR sensor model 215. The LSPR sensor model 215 (or R below) models the reflectance/absorbance spectrum of the LSPR sensor 110. The reflectance/absorbance spectrum is dependent on the peak wavelength 210.

In one example, the reflectance/absorbance spectrum of the LSPR sensor 110 may be modeled as a function of the form:

$$R = a + bf ( c , d ) ,$$

where a, b, c and d are parameters of the model and f is a known function involving the peak wavelength 210.

The maximum value of R comes from the peak of f(c,d) which encompasses the peak wavelength 210 and will depend on parameters a, b, c and d. In an embodiment, the parameters a and b represent an arbitrary base and scale respectively.

In a preferred embodiment, the function f(c,d) is determined using a computer to calculate the Mie scattering theory applied to AuNPs. Mie scattering theory is a solution to the Maxwell equations which describes the scattering of an electromagnetic wave by a homogeneous spherical particle having a refractive index (Ri) different from that of the medium surrounding it. Mie theory can also be applied to non-homogeneous core-shell spheres. In the LSPR case, the AuNP forms the core, while the ligand and bound analyte together form the shell. As the shell thickness or refractive index changes, Mie theory predicts a change in the scattering properties of the nanoparticles. It also predicts a shift in the wavelength peak. The parameters c and d represent shell thickness and shell refractive index or some functions of these quantities.

In another embodiment, the function f is modeled as a log-normal function. A log-normal function is the probability distribution function of a random variable where the logarithm of that function is normally distributed. So, if Y is normally distributed, then X, such that Y=ln(X) is log-normally distributed. Function ln( ) represents the natural log. The mathematical expression for log-normal function is as follows:

$$p ( ω ) = \frac{1}{w σ \sqrt{2 π}} {e}^{\frac{- {( ln ( ω ) - μ )}^{2}}{2 {σ}^{2}}}$$

For log-normal function, it's maxima can be found at:

$${ω}_{max} = {e}^{( μ - {σ}^{2} )}$$

Considering parameters c=μ and d=σ, the function f can be re-written as

$$f ( c , d ) = \frac{1}{ω d \sqrt{2 π}} {e}^{\frac{- {( ln ( ω ) - c )}^{2}}{2 {d}^{2}}}$$

In an embodiment, the parameters of f are defined as: c=e^{μ} and d=e^{σ}. The function f can then be written as

$$f ( c , d ) = \frac{1}{ω ln ( d ) \sqrt{2 π}} {e}^{\frac{{- ( ln ( ω ) - c ) )}^{2}}{2 {( ln ( d ) )}^{2}}}$$

A source spectrum 220 represents the spectrum provided by the illumination source 115 of the LSPR sensor 110. The LSPR sensor model reflectance/absorbance spectrum R 215 gets multiplied with the source spectrum 220 to give a reflected/absorbed spectrum of light, r. Thus, reflected/absorbed spectrum of light is given by:

$$r = S ( a + bf ( c , d ) ) ,$$

where S represents the illumination source spectrum 220.

The effects of dispersive optics 125 are modeled by a diffraction model 225. In one example, the dispersive optics is modeled as a convolution. For example, the convolution kernel may be modeled as the area of a circle convolved with a 1D Gaussian function. The circle represents the exit surface of the fiber optics and the Gaussian represents the dispersion due to the optical components. Thus, the effect of the dispersive optics on the reflected/absorbed spectrum of light can be represented as a 2D convolution operator G:

$$m = G * S ( a + bf ( c , d ) ) , \qquad ( 1 )$$

where G represents the convolution kernel of the dispersive optics 225. In an embodiment, G is modeled as the convolution of a discrete 2D function of the area of a circle, which is 1 inside a circle and 0 outside the circle, with a Gaussian distribution.

The parameter m represents the model spectral image as opposed to the actual spectral image recorded by the imaging sensor 130. Due to physical limitations as well as the limitations of the imaging sensor 130, the model spectral image m, in equation (1) above, is perturbed by an imaging noise 230. The imaging noise 230 includes one or more of photon noise, quantization noise, dark noise, additive white noise, and the like. Referring to FIG. 2, m may be represented very generally by: m=N(G(S(R)))), where the imaging noise (N, 230) is a function of the diffraction model (G, 225), which itself is a function of the source spectrum (S, 220) which is a function of the sensor model (R, 215).

In one example, the imaging noise 230 includes photon noise only. The number of photons falling on each pixel of the imaging sensor 130 is different in each exposure interval. This variation, also called the photon noise, typically follows a Poisson distribution. For a Poisson distributed photon noise and an expected pixel value of mi, the probability of observing pixel value x; in data is given by:

$$p ( {x}_{i} \vert {m}_{i} ) = \frac{{m}_{i}^{\frac{{x}_{i}}{k}} {e}^{- {m}_{i}}}{\frac{{x}_{i}}{k} !} ,$$

where k is the number of digital levels per photon—also known as analog gain of the imaging sensor 130.

The dispersed and noise perturbed reflected/absorbed light data may be stored in the imaging sensor 130 as a spectral image 235.

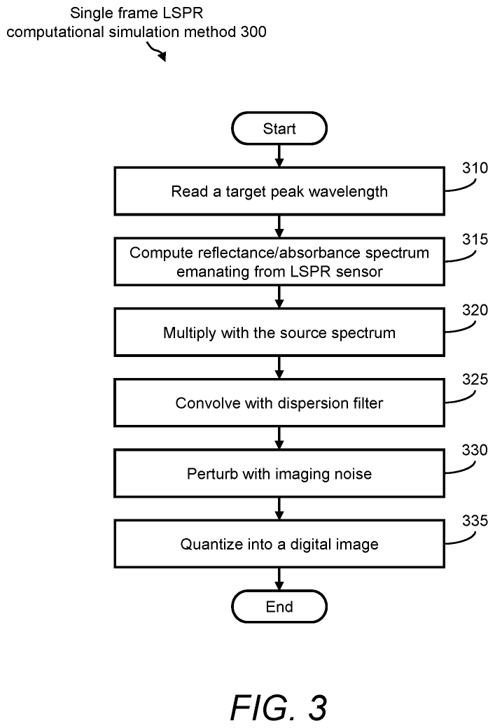

FIG. 3 is a flow diagram of an example of a method 300 of computationally simulating a single frame of an LSPR spectrometer system, such as the LSPR spectrometer system 100 shown in FIG. 1. The image processing method 300 includes a step 310 of reading the target peak wavelength of the reflectance/absorbance spectrum. For example, the target peak wavelength is supplied by a binding kinetics reaction simulator. After reading the target peak wavelength, the corresponding reflectance/absorbance spectrum is computed in a step 315. For example, step 315 may include finding the parameters of the LSPR sensor model 215 and using the parameters to compute reflectance/absorbance R, such that:

$$R = a + bf ( c , d ) ,$$

where a, b, c and d are parameters of the model and f is a known function. Step 315 may involve finding the parameters such that the maxima of R is close to the target peak wavelength.

In one example, the function f may be determined using the Mie scattering theory. In this example, the parameters c and d represent shell thickness and shell refractive index or some functions of these quantities.

In another example, the function f may be modeled as a log-normal function. In this example, the parameters c and d represent u and o respectively.

Reflectance/absorbance is converted to reflected/absorbed light by multiplying it with the source light spectrum in a step 320. Next, a step 325 includes convolution of the reflected/absorbed light with the convolution kernel G of the diffraction model 225. For example, in step 325, a 1D spectrum is converted to 2D spectral data. The 2D spectral data is perturbed by adding imaging noise in a step 330. The imaging noise includes one or more of photon noise, quantization noise, dark noise, additive white noise, and the like. In one example, the imaging noise includes photon noise only. The noise perturbed 2D spectral data is quantized in a step 335 and then converted to a 2D digital image.

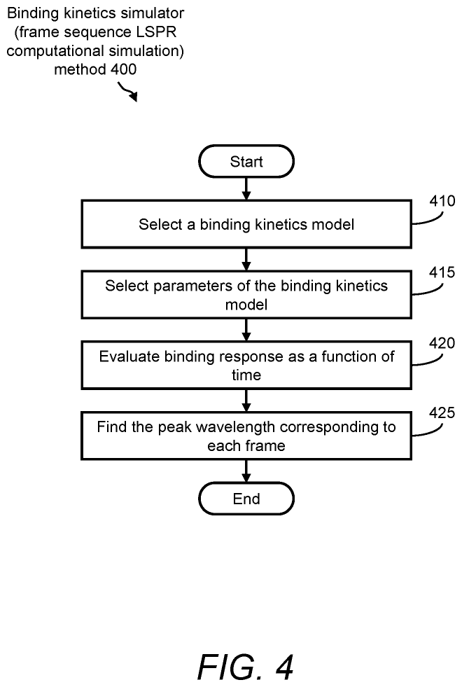

FIG. 4 is a flow diagram of an example of a method 400 of computationally simulating a frame sequence of an LSPR spectrometer system, such as the LSPR spectrometer system 100 shown in FIG. 1. In this example of the method 400, the frame sequence corresponds to temporal binding of a ligand and an analyte.

The method 400 begins by a step 410 of selecting a binding kinetics model. Binding kinetics models may include, but are not limited to, Langmuir 1:1 model, Langmuir 1:1 model with mass transport limitations, Langmuir 1:1 model with drift, bivalent analyte model, two-state conformation model, heterogeneous ligand model, heterogeneous analyte model, and the like. Each of these models are parametric Ordinary Differential Equation (ODE) systems. Next, a step 415 includes selecting parameters of the model chosen in step 410. A set of parameters represents the binding kinetics of some real or imagined analyte-ligand pair. Given the ODE model and its parameters, the binding response is evaluated in a step 420 as a function of time. In one example, a closed form solution for the ODE model is evaluated. In another example, the ODE model is integrated numerically to evaluate the binding response. Each binding response value corresponds to a peak reflectance/absorbance wavelength of the LSPR spectrometer system. The peak wavelength at discrete time instances is computed in a step 425. In one example, the discrete-time peak wavelength value represents the peak wavelength 210 of mathematical model 200. In one example, the target peak wavelength for step 310 of method 300 of FIG. 3 is supplied using the peak value computed in step 425 of method 400 of FIG. 4.

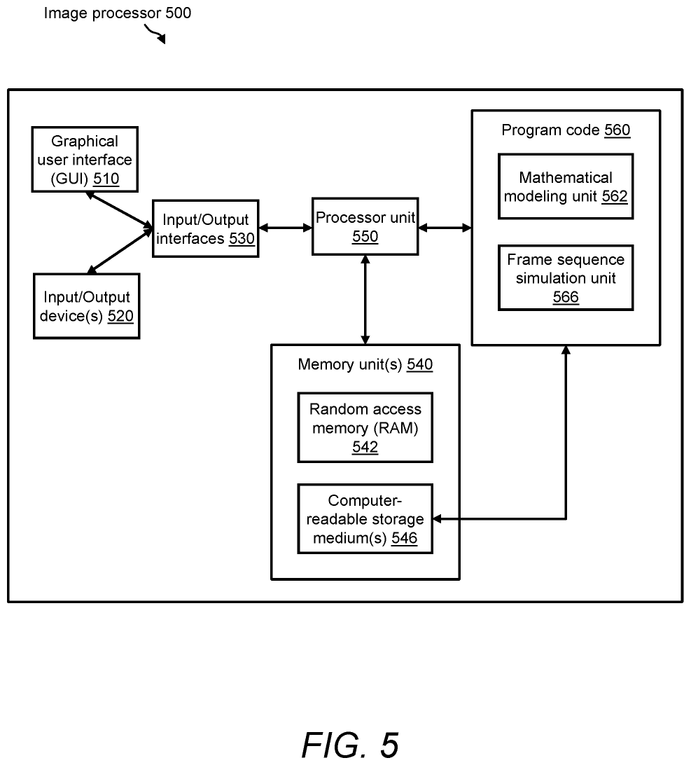

FIG. 5 is a block diagram of an example of an image processor 500 for executing computational simulations in accordance with the invention.

In one embodiment, the image processor 500 may be a general purpose computer system containing instructions for executing computational simulations. The image processor 500 may consist of components in a single computer or computer system, or its functions and components may be distributed among multiple physical structures. As illustrated in FIG. 5, the image processor 500 may further comprise one or more processor units 550, memory units 540, input and/or output interfaces 530 and program code 560 containing instructions that can be read and executed by the processor 550. One or more input interfaces 530 may connect memory units 540 and processor units 550 to one or more input devices 520, such as a keyboard, mouse, touch screen, voice-activated systems or other suitable device(s). Thus, input devices 520 may allow users to communicate commands to one or more processors. One such example command is the execution of program code 560. Another example command may involve modifying the input parameters of a mathematical model to modify the maximum likelihood peak fitting algorithm, for example, and corresponding simulation output results. Output interfaces 530 may be connected to memory units 540, processor units 550 and graphical user interfaces (GUIs) 510. Thus, output interfaces 530 enable the image processor 500 to transmit data from the one or more processors to output devices. One such exemplary transmission includes spectral images created as a result of the computational simulation methods described herein. The graphical user interface 510 may also function as an input interface as, for example, by comprising touch-sensitive displays or screens.

Memory units 540 may include conventional semiconductor random access memory (RAM) 542 or other forms of memory known in the art; and one or more computer-readable storage media 546, such as a hard drive, flash drive, optical drive, etc.

In an embodiment of the disclosure, program code 560 may be stored on computer-readable storage media 546. As illustrated in FIG. 5, program code 560 further includes a mathematical modeling unit 562 containing instructions for computing an absorbance/reflectance spectrum in accordance with the methods disclosed herein and a frame sequence simulation unit 566 containing instructions for computationally simulating a frame sequence of an LSPR spectrometer system corresponding to temporal binding of a ligand and an analyte.

Combining the physical LSPR spectrometer system 100 of FIG. 1, the mathematical model 200 of an LSPR spectrometer system of FIG. 2, the single frame LSPR computational simulation method 300 of FIG. 3, the frame sequence LSPR computational simulation method 400 of FIG. 4, and the image processor 500 of FIG. 5, a physics-aware computational simulation of an LSPR spectrometer may be generated leading to the following benefits:
1. Numerous data sets can be created, which would augment those produced on the actual hardware. This would allow testing of the LSPR data analysis software with greater variety of data.
2. The simulated datasets are devoid of any systemic noise (physico-chemical noise) that could be present in hardware datasets. Thus, it would be possible to measure the performance of the developed LSPR spectrometer data analysis algorithms with respect to purely statistical noise.
3. The simulations can be used to judge performance of existing and new spectrometers. The effect of using different spectrometer systems and different variants of the subcomponents of the system can be analyzed and optimal configurations may be chosen. The simulations can drive the production of novel spectrometers tailor-made for a particular application, such as the sensing of absorbed/reflected AuNP spectrum.

We claim:

1. A method for computationally simulating an LSPR spectrometer system, the method comprising:
a. reading a target peak wavelength;
b. using a mathematical model of the LSPR spectrometer system to compute an absorbance/reflectance spectrum;
c. using the mathematical model of the LSPR spectrometer system and an illumination source spectrum to compute an absorbance/reflectance spectrum of optical dispersion; and
d. perturbing the absorbed/reflected spectrum with optical dispersion imaging noise to create a noise perturbed spectrum.

2. The method of claim 1 wherein the noise perturbed spectrum is stored as a 2D image.

3. The method of any one of the preceding claims, wherein the absorbance/reflectance spectrum is computed using Mie theory.

4. The method of any one of the preceding claims, wherein the absorbance/reflectance spectrum is computed using a log-normal function.

5. The method of any one of the preceding claims, wherein the optical dispersion imaging noise is modeled using a 2D convolution.

6. The method of any one of the preceding claims, wherein the imaging noise is photon noise.

7. The method of any one of the preceding claims, wherein the target peak wavelength is computed using a binding kinetics reaction simulator.

8. A method for computationally simulating a binding kinetics reaction, the method comprising:
a. choosing a binding kinetics model and parameters for the binding kinetics model;
b. using the binding kinetics model to compute a binding response as a function of time;
c. discretizing the binding response into a plurality of discrete time instances; and
d. finding the peak wavelength corresponding to each discrete time instant.

9. The method of claim 8, wherein the binding kinetics model is Langmuir 1:1

10. The method of claim 8, wherein the binding kinetics model is Langmuir 1:1 with mass transport limitations.

11. The method of claim 8, wherein the binding kinetics model is Langmuir 1:1 with drift.

12. The method of claim 8, wherein the binding kinetics model is a two-state conformation model.

13. The method of claim 8, wherein the binding kinetics model is a bivalent analyte model.

14. The method of claim 8, wherein the binding kinetics model is a heterogeneous analyte model.

15. The method of claim 8, wherein the binding kinetics model is a heterogeneous ligand model.

16. The method of claim 8 wherein the binding response is computed using numerical integration of the binding kinetics model.
